(19) United States
(12) Patent Application Publication
(10) Pub. No.: US 2010/0137810 A1
(43) Pub. Date: Jun. 3, 2010

(54) SAFETY GUARDS FOR SYRINGE NEEDLE

(76) Inventors: Margam Chandrasekaran, Singapore (SG); John Ming Shyan Yong, Singapore (SG); Hs Ramanath, Singapore (SG); Zelia Tay, Singapore (SG)

Correspondence Address: DICKSTEIN SHAPIRO LLP, 1825 EYE STREET NW, Washington, DC, 20006-5403, US

(21) Appl. No.: 12/595,360

(22) PCT Filed: Mar. 24, 2008
(86) PCT No.: PCT/SG08/00091

§ 371 Date: Oct. 9, 2009

(30) Foreign Application Priority Data
Apr. 11, 2007 (SG) 200702595-0

(51) Int. Cl. A61M 5/32

(52) U.S. Cl. 604/198

(57) ABSTRACT

A safety guard (10) for a needle has a slider (22) for slideably encasing the needle; a moveably traveler (34) that slides over and with the slider and is in sliding engagement with and within a housing (14). A resilient member (80) within the housing biases the slider distally to cover the needle. Radial recesses (26a, 26b) on the outer surface of the slider are engaged by cantilevered snaps (38) at the distal end of the traveller. Once the snaps are engaged they are prevented from moving proximally with respect to the slider, but can move distally from one set of recesses (26a) to another (26b).

### TECHNICAL FIELD

This invention relates generally to a safety guard for a syringe needle and more particularly, though not exclusively, to a safety guard for hypodermic needle assemblies able to shield a tip of the needle before, during and after use.

### BACKGROUND

Medical care of individuals requires the widespread use of needles for taking blood samples, intravenous drug delivery, and the introduction or removal of other fluids via canula, needles, or syringes. In the current context, the use of hypodermic needles to deliver plasma, anaesthetics, or other medications has become commonplace in medicine, science, veterinary medicine, and biotechnology. The use of a hypodermic needle typically involves first inserting a needle into the patient, injecting a substance or withdrawing a substance as required, and then removing the needle from the patient. In most applications, the withdrawn and contaminated needle must be handled very carefully during disposal to avoid ‘needle stick’ injury.

To help prevent health care workers from being injured, guards have been developed to block the tips of these needles after use. Needle stick protection for medical professionals has become of particular importance in recent years because of the prevalence of potentially fatal infectious diseases, such as Acquired Immune Deficiency Syndrome (AIDS) and hepatitis, and that can be transmitted by the exchange of bodily fluids through inadvertent wounds caused by accidental needle tip pricks after withdrawal from infected patients. Accordingly, many kinds of needle protection devices are available for providing post injection needle stick protection.

Devices with integrated safety features which have been introduced to provide protection against punctures by hypodermic needles fall into three basic categories: those which withdraw the needle into the barrel of the syringe after use; those with a hinged needle guard which rotates into position over the needle; and those with a sliding shield which moves along the needle shaft and covers its tip. The guards may be manually moved into position by the user or given mechanical assistance such as, for example, by the use of springs or suction.

A key problem with manually activated designs is that they require the user to either slide or apply the needle shield to the tip of the needle by hand, significantly raising the risk of unintentional contact with the needle tip. There is also a danger of the user forgetting to activate the device. In addition, many of these needle guards interfere with the ability to use the syringe with single hand so a powered device is desirable.

Even amongst powered devices, a pervasive problem with most existing designs is that they provide no integrated facility for covering the needle tip before and during loading of the drug such as, for example, from a vial. The safety features are only activated following use. This increases the likelihood of needle stick injury. To solve this issue, additional safety caps are often provided but these increase the cost of the devices and introduce an additional user action—removal of the safety cap—where accidental puncturing of the skin can occur.

There are a few designs which have attempted to provide protection before and after use. These are generally operated manually or are spring biased to extend a tubular shield and enclose the needle canula. These systems mainly include spiral or complicated channels cut into the shield which guide it during extension and lock it in the extended position. A problem with these designs is that the track systems may not always be reliable.

Also, trigger-based systems may be activated unintentionally. This may happen in circumstances such as, for example, if the needle contacts a bone in the patient. This leads to discomfort for the patient and potentially serious danger from air bubbles being introduced into the blood stream due to cavitation. Furthermore, the activation of the trigger-based system is often unintuitive for users, leading to potential errors in operation.

### SUMMARY

According to an exemplary aspect there is provided a safety guard for a needle of a syringe. The safety guard comprises a moveable slider for slideably encasing the needle and a moveable traveller for sliding engagement over and with the slider and sliding engagement with and within a housing. At least one resilient member is within the housing for biasing the slider distally.

The traveller may be attachable to the slider. The traveller may be attachable to the slider by radially directed projections engageable in recesses to allow distal movement and prevent proximal movement of the traveller with respect to the slider when the traveller is attached to the slider. The projections may be configured to be a snap fit in the recesses. The projections may comprise at least one cantilever snap on the traveller and the recesses comprise corresponding recesses in the slider. The recesses in the slider may comprise through holes in the slider each having a distal side angled to provide a distally sloping surface. The recesses in the slider may comprise proximal recesses and distal recesses. The proximal recesses and the distal recesses may be axially spaced along the slider. The cantilever snap on the traveller may have an angled distal face such that the traveller is allowed to move distally along the slider from the proximal recesses to the distal recesses while the traveller is prevented from moving proximally when the cantilever snap is engaged with a recess.

The projections may comprise at least one cantilever snap on the slider and the recesses comprise corresponding recesses in the traveller. The recesses in the traveller may comprise rectangular through holes in the traveller each having a proximal side angled to provide a proximally sloping surface. The recesses in the traveller may comprise proximal recesses and distal recesses, the proximal recesses and the distal recesses being axially spaced along the traveller. The cantilever snap on the slider may have an angled proximal face such that the slider is allowed to move proximally along the traveller from the distal recesses to the proximal recesses while the traveller is prevented from moving proximally when the cantilever snap is engaged with a recess.

The projections may comprise an annular snap on the traveler. The recesses may comprise annular recesses in the slider. Alternatively or additionally, the projections may comprise an annular snap on the slider. The recesses may comprise annular recesses in the traveller.

The traveller may be restrainable within the housing by further projections that engage in further recesses to allow distal movement and prevent proximal movement of the traveller with respect to the housing when the traveller is proximally restrained within the housing. The further projections may be configured to be a snap-fit in the further recesses. The further projections may comprise at least one further cantilever snap on the traveller and the further recesses comprise corresponding recesses on the housing. The recesses in the housing may comprise through holes in the housing. The recesses in the housing may be axially spaced along the housing. The further cantilever snap on the traveller may have an angled distal face such that the traveller is allowed to move distally within the housing from one recess to an adjacent distal recess in the housing while the traveller is prevented from moving proximally when the cantilever snap is engaged with a recess.

The further projections may comprise an annular snap on the traveller and the further recesses comprise corresponding annular recesses in the housing.

The slider may further comprise lateral protrusions for engaging proximally adjacent portions on the traveller to prevent proximal movement of the slider. The projections and the further projections may be axially aligned.

The safety guard may further comprise a cap for retaining at least a portion of the slider and at least a portion of the traveller within the housing.

The slider may have at least one external key for sliding engagement with a branched keyway in the traveller. The branched keyway in the traveller may comprise an angled portion for effecting axial and rotational proximal movement of the slider, and an axial portion having a recess for retaining the external key on the slider to lock the slider with the traveller.

The traveller may have at least one external key for slideable engagement with a branched keyway in the housing. The branched keyway in the housing may comprise an angled portion for effecting axial and rotational proximal movement of the traveller, and an axial portion having a recess for retaining the external key on the traveller to lock the traveller with the housing.

The safety guard may further comprise a second resilient member for biasing the traveller distally.

A proximal portion of the housing may be adapted for engaging the syringe.

### BRIEF DESCRIPTION OF THE DRAWINGS

In order that the invention may be fully understood and readily put into practical effect there shall now be described by way of non-limitative example only preferred embodiments of the present invention, the description being with reference to the accompanying illustrative drawings.

In the drawings:

FIG. 1 is a schematic side view of an exemplary embodiment attached to a syringe

FIG. 2 is a schematic cross-sectional view of an exemplary embodiment assembled with a needle in its initial position;

FIG. 3 is schematic cross-sectional view of the exemplary embodiment of FIG. 1 in its intermediate position;

FIG. 4 is a schematic cross-sectional view of the exemplary embodiment in its final state; and

FIG. 5 is a schematic cross-sectional view of another exemplary embodiment assembled with a needle in its initial position;

FIG. 6 is a schematic cross-sectional view of the exemplary embodiment of FIG. 4 rotated 90 degrees;

FIG. 7 is an exploded perspective view of the assembly of FIGS. 1 to 6;

FIG. 8 is a schematic cross-sectional view of the exemplary embodiment of FIG. 1 with the needle exposed;

FIG. 9 is a schematic cross-sectional view of the exemplary embodiment of FIG. 2 with the needle exposed; and

FIG. 10 is an exploded perspective view of an alternative embodiment of the assembly of FIG. 7;

FIG. 11 is a schematic cross-sectional view of yet another exemplary embodiment assembled with a needle in its initial position.

FIG. 12 is a schematic cross-sectional view of the exemplary embodiment of FIG. 10 with the needle exposed;

FIG. 13 is a schematic cross-sectional view of the exemplary embodiment of FIG. 11 in its final state;

FIG. 14 is a side view of yet another exemplary embodiment;

FIG. 15 is a close-up schematic view of a branched keyway and key of the embodiment of FIG. 14 in an initial position; and

FIG. 16 is a close-up schematic view of the branched keyway and key of the embodiment of FIG. 15 in a final position.

### DETAILED DESCRIPTION OF EXEMPLARY EMBODIMENTS

The exemplary embodiment is a safety guard for a needle of a syringe such that, when attached to the syringe, the sharp distal needle tip is shielded during the majority of typical user actions, yet remains under the full control of the user.

Typical user actions include the removal of the device from its packaging, attachment to a syringe, loading of medicament into the device, temporary storage while awaiting use, transportation to or from a patient, injection of medicament into a patient, withdrawal of fluid such as blood from a patient, temporary storage following use and permanent device disposal.

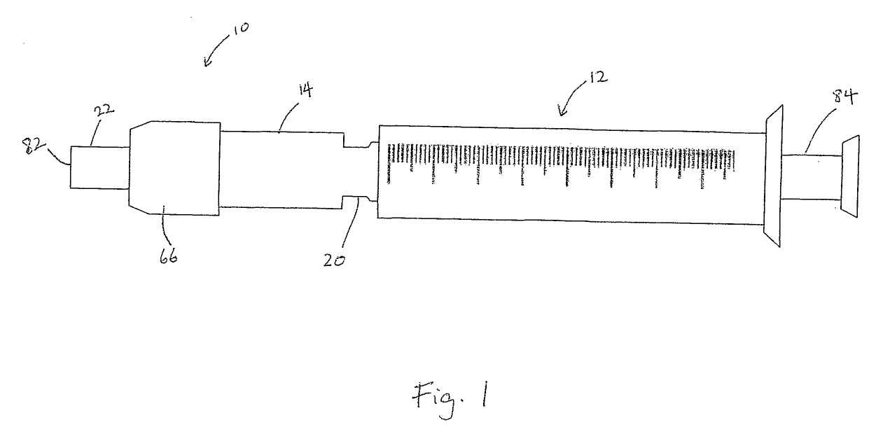

As shown in FIG. 1 there is a guard 10 having a housing 14 for releasable attachment to a typical plastic syringe 12. This may be by the use of a standard attachment mechanism such as, for example, a Luer lock 20, as shown. The Luer lock 20 is preferably integral with the housing 14.

FIGS. 2, 3 and 4 show the guard 10 being integral with a needle 16 having a needle tip 18 distal from the Luer lock 20. However, the guard 10 may be a separate component from the needle that is securely and non-releasably attachable to the needle.

The guard 10 has a moveable slider 22 that is co-axial with the needle 16. The slider 22 slideably encases the needle 16. A portion of the slider 22 extends distally beyond the housing 14. The slider 22 has a generally cylindrical body 24. On the outer surface of the body 24 are recesses 26a and 26b spaced apart from each other along the body 24. The recesses 26a, 26b can each be in the form of a pair of two rectangular recesses diametrically opposite each other on the cylindrical body 24. The body 24 may be hollow, in which case the recesses 26a, 26b can be pairs of rectangular through holes in the body 24. Although the recesses have been described as being rectangular, they can also be of any other suitable shape. The sides of the recesses 26a, 26b can extend generally radially of the body 24 or, more preferably, have one side (the proximal side) that extends generally radially of the body 24, and one side (the distal side) that is angled relative to the body to provide a sloping surface that is angled towards the distal end of the body 24.

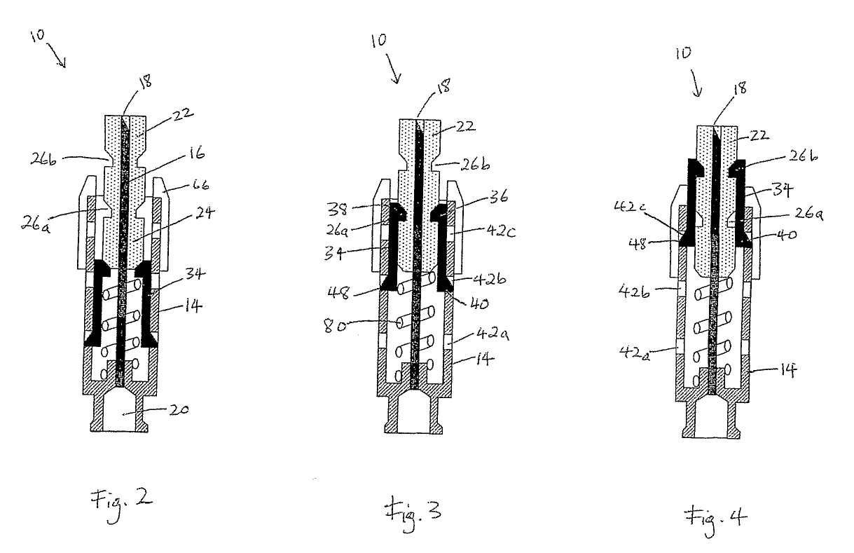

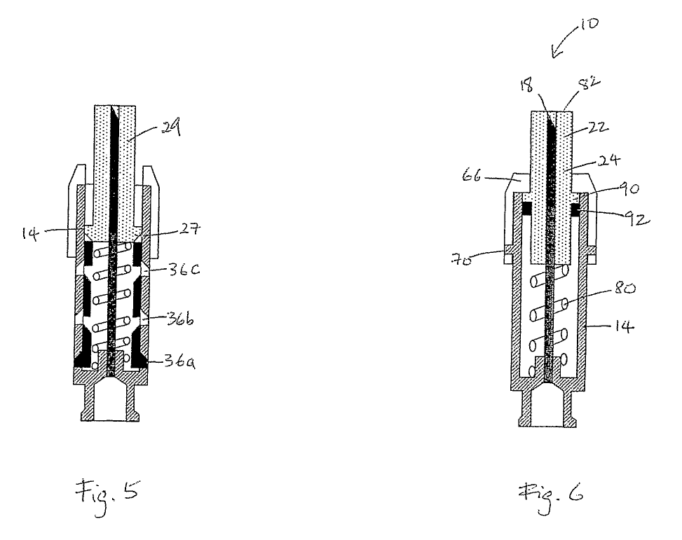

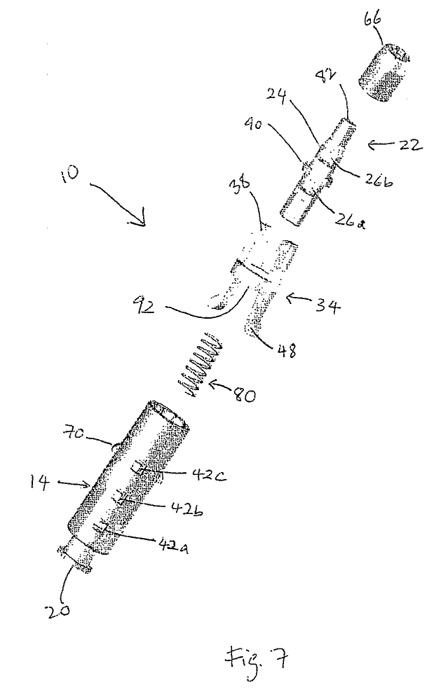

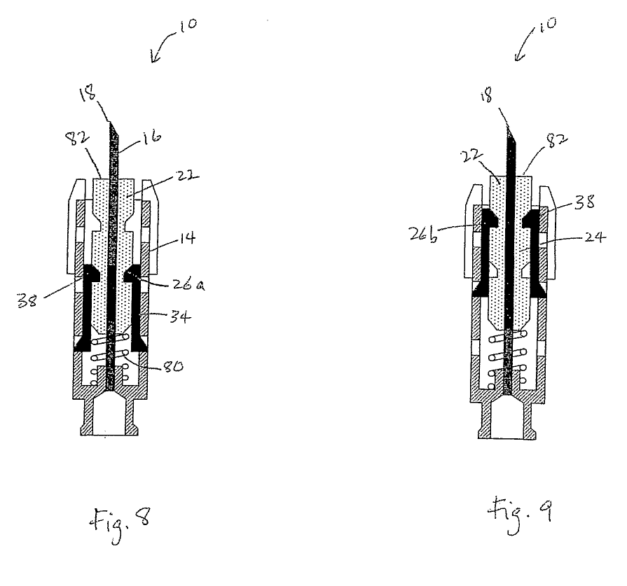

Also co-axial with the needle 16 and partially surrounding the slider 22 is a generally hollow traveller 34 for sliding engagement with the slider 22. The distal end 36 of the traveller 34 is adapted to engage the recesses 26a, 26b on the body 24, so as to attach the traveller 34 to the slider 22. Where the recesses 26a, 26b are rectangular recesses, the distal end 36 of the traveler 34 has projections that, as shown, are in the form of a pair of cantilever snaps 38 at the distal end of the traveler 34. The snaps 38 are diametrically opposite each other and extend radially inwardly of the traveller 34. As can be seen in FIGS. 2, 3 and 4, the snaps 38 are designed such that the traveller 34 is prevented from moving proximally with respect to the slider 22 once the snaps 38 have engaged either pair of rectangular recesses 26a, 26b. However, the snaps 38 have an angled distal face such that the traveller 34 can move distally along the body 24 from one pair of recesses 26a (shown in FIG. 3) to the next pair of recesses 26b (shown in FIG. 4). Preferably, the engagement of the snaps 38 with recesses 26a, 26b is in the form of a snap fit.

Although it has been described above that the recesses are in the slider while the snaps are on the traveller, in another embodiment as shown in FIG. 5, recesses 36a, 36b, 36c could be in the traveller 39 while the snaps 27 are on the proximal end of the slider 29. This would provide the same releasable snap fit configuration to prevent proximal movement of the traveller with respect to the slider once the snaps 27 have engaged the recesses 36a, 36b, and 36c.

Alternatively, instead of using cantilever snaps fitting in recesses and operating between the slider 22 and the traveller 34, annular snaps could be used. Instead of being pairs of rectangles, the recesses 26a, 26b could be annular recesses in the body 24. Likewise, the distal end 36 of the traveller 34 could comprise an annular snap instead of the cantilever snaps 38. The reverse of having annular recesses in the traveller 34 and annular snaps on the proximal end of the body 24 would also achieve the same effect.

As shown in FIGS. 2 to 4, the proximal end 40 of the traveller 34 is adapted to engage recesses 42a, 42b, and 42c on the housing 14, so as to restrain the traveller 34 within the housing 14. Where the recesses 42a, 42b, 42c are rectangular recesses, the proximal end 40 of the traveller has outwardly-directed projections that, as shown, are in the form of a second pair of cantilever snaps 48 that are diametrically opposite each other and proximally of and preferably axially aligned with the snaps 38. The second snaps 48 are designed such that the traveller 34 is prevented from moving proximally with respect to the housing 14 once the second snaps 48 have engaged a pair of rectangular recesses 42a, 42b, and 42c. However, each of the second snaps 48 have a distal face that is angled such that the traveller 34 can move distally along the housing 14 from one pair of recesses to the next. This is further facilitated by having the distal edges of the rectangular recesses 42a, 42b, and 42c slope inwards radially and distally.

Alternatively, instead of using cantilever snaps fitting in recesses and operating between the traveller 34 and the housing 14, annular snaps could be used. Instead of being pairs of rectangles, the recesses 42a, 42b, 42c could be annular recesses in the housing 14. Likewise, the proximal end 40 of the traveller 34 could comprise an annular snap instead of the cantilever snaps 48.

When the snaps 38 and 48 on the traveller 34 are simultaneously engaged with the most distal recesses 26b and 42c on the slider 22 and the housing 14 respectively as shown in FIG. 4, the slider 22 is prevented from sliding proximally with respect to the needle 16 by means of lateral protrusions 90 on the body 24 of the slider 22 engaging proximally adjacent portions 92 on the traveller 34 as shown in FIG. 6. When the guard is in this configuration, the needle tip 18 can no longer be exposed. A perspective view of the slider 22 and traveller 34 can be seen in FIG. 7 which shows an exploded view of the guard 10.

Securely attachable to the distal end of the housing 14 is an end cap 66 which is able to slideably engage with the slider 22. The cap 66 can be fixedly attached to the distal end of the housing 14 by any suitable attachment mechanism including, but not limited to, a buckle engaging a clip 70. The cap 66 is further adapted to retain at least a portion of the slider 22 and the traveller 34 within the housing 14. When in the position shown in FIG. 6, the lateral protrusions 90 on the body 24 of the slider 22 engage the proximal side of cap 66 to prevent the slider 22 from being removed from the guard 10.

A flexible, resilient member 80 such as, for example, a compression spring is co-axial with and surrounds the needle 16 within the housing 14. The resilient member 80 may be of any other suitable form including, but not limited to, foam, or be an integral part of the slider 22. The spring 80 engages and extends between the proximal end of the slider 22 and the proximal end of the housing 14. The spring 80 biases the slider 22 distally.

To use the safety guard 10, the user removes it from its storage or packaging and, in the Luer lock form shown, attaches it to the syringe 12 by grasping the housing 14, placing the guard 10 with needle in position and rotating until a seal is made between the syringe 12 and the housing 14. This assembly will now be referred to as a hypodermic syringe. In the original position, the needle tip 18 is within the slider 22 and does not extend beyond the distal end 82 of the slider 22.

To charge the hypodermic syringe with medicament, the distal end 82 of the slider 22 is pressed against the membrane of a typical medicament vial. The pressure of the membrane on the distal end 82 causes the slider 22 to slide axially and proximally relative to the needle 16 against the force of the spring 80, while the traveller 34 simultaneously slides over the slider 22 as shown in FIG. 8. This causes the needle tip 18 to extend beyond the distal end 82 and penetrate the membrane. The maximum extent of movement is when the snaps 38 on the traveller 34 engage the recesses 26a on the body 24 of the slider 22, and second snaps 48 engage the recesses 42a, thereby attaching the traveller 34 to the slider 22 as shown in FIG. 8. The user will know this has occurred by an audible ‘click’ when the snaps 38 engage the recess 26a and second snaps 48 engage recesses 42a. This also allows a large portion of the needle 16 to extend beyond the distal end 82 and thus be exposed. The user then pulls back the syringe plunger 84, drawing fluid through the needle 16 into the syringe 12. When charged to the desired volume, the hypodermic syringe is removed from the vial. The force of spring 80 is sufficient to cause second snaps 48 to disengage from recesses 42a due to the sloping distal faces. As such, the slider can 22 returns to its original position under the influence of the spring 80 to cover the needle tip 18 as shown in FIG. 3. As the slider 22 returns to its original position, the traveller 34 moves together with the slider 22 distally within the housing 14 into an intermediate position as shown in FIG. 3 due to the engagement of snaps 38 in recesses 26a. The second snaps 48 engage the recesses 42b, again providing an audible click. The hypodermic syringe can then be transported to the patient or safely stored for a short period until the injection of the medicament is required. The hypodermic syringe can be charged with multiple medicaments if the user takes care not to apply so much pressure as to cause the snaps 38 on the traveller 34 to engage the recess 26a on the body 24 of the slider.

When the injection is required, the distal end 82 of the slider 22 is pressed against the skin of the patient at the injection site. The pressure of the skin on the distal end 82 once more causes the snaps 38 to disengage from recesses 26a due to the sloping distal faces and thus the slider 22 can slide axially and proximally relative to the needle 16 against the force of the spring 80, while the traveller 34 simultaneously slides over the slider 22 as shown in FIG. 9. This causes the needle tip 18 to extend beyond the distal end 82 and thus penetrate the skin. The maximum extent of movement is when the snaps 38 on the traveller 34 engage the recess 26b on the body 24 of the slider 22, once more attaching the traveller 34 to the slider 22 as shown in FIG. 9. The user will know this has occurred by an audible ‘click’ when the snaps 38 engage the recesses 26b. The user then pushes the syringe plunger 84, injecting fluid from the syringe through the needle 16 into the patient. When discharged, the hypodermic syringe is removed from the patient. The force of spring 80 is sufficient to cause second snaps 48 to disengage from recesses 42b due to the sloping distal faces. As such, the slider 22 returns to the position shown in FIG. 4 under the influence of the spring 80 to cover the needle tip 18. As the slider 22 returns to its original position, the traveller 34 moves together with the slider 22 due to engagement of snaps 38 in recesses 26b. The movement is distally within the housing 14 into the state as shown in FIG. 4. In this state, the guard 10 is locked in position as shown in FIG. 6, preventing re-use of the needle 16 as well as needle stick injury during disposal. The hypodermic syringe can then be stored and disposed of in accordance with known, correct procedures.

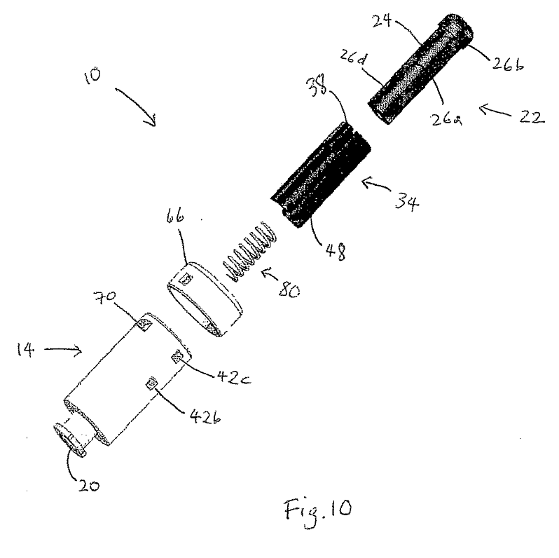

An alternative embodiment of the guard 10 is shown in FIG. 10. Reference numerals of corresponding features in FIG. 7 and FIG. 10 have been kept the same for ease of understanding since the corresponding features function similarly. In FIG. 10, the snaps 38 and the second snaps 48 on the traveller 34 are circumferentially offset, unlike in FIG. 7 where they are axially aligned. Also, the slider 22 comprises additional recesses 26d for engaging the snaps 38 in the original position. The housing 14 only has recesses 42b and 42c for engaging the second snaps 48 in the intermediate and final positions. These are variations in design and do not affect the operating principle of the guard 10.

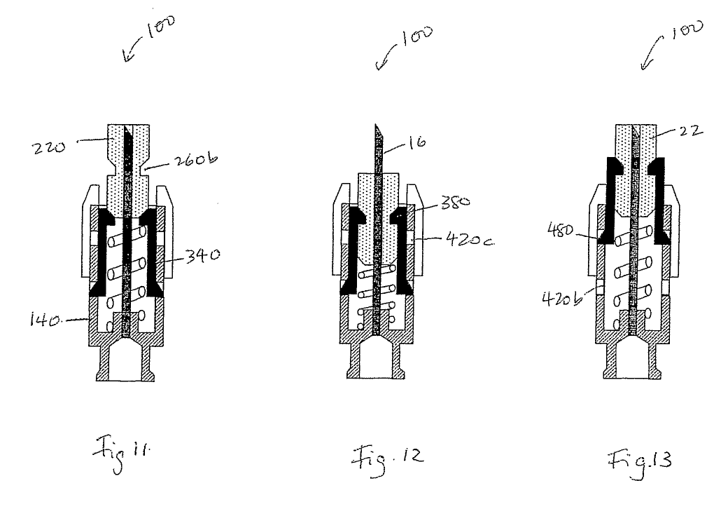

Another embodiment of the guard is depicted in FIGS. 11 to 13. In this embodiment, the guard 100 is for attaching to a syringe already preloaded with medicament (not shown). In use, no charging of the syringe is required. Only injection will take place. The guard 100 is similar to the guard 10. The only differences are that the slider 220 only needs one pair of recesses 260 (corresponding to recesses 26b earlier described) for engaging the snaps 380 on the traveller 340, and the housing 140 only needs two pairs of recesses 420b, 420c (corresponding to recesses 42b, 42c earlier described) for engaging the snaps 480 on the traveller.

It should be noted that the embodiments of the guard 10 shown in FIGS. 2 to 10 can also be used for a preloaded syringe. This can be achieved by ensuring during assembly of the guard 10 that the slider 22, traveller 34 and housing 14 are packaged such that the second pair of cantilever snaps 48 are engaged with the recesses 42b in the housing 14, and the cantilever snaps 38 are engaged with the recesses 26a in the slider 22 (as shown in FIG. 3).

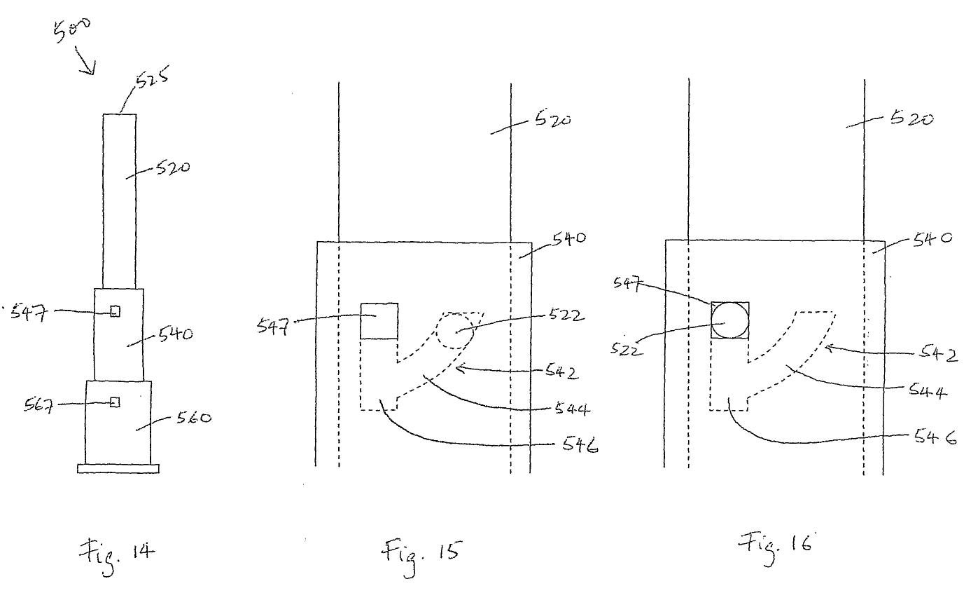

Another embodiment of the guard is depicted in FIGS. 14 to 16. As shown, the guard 500 comprises a moveable slider 520 for encasing a needle (not shown). A traveller 540 slideably engages the slider 520. The traveller also slideably engages a housing 560. At least one resilient member such as a spring (not shown) in the housing 560 biases the slider 520 distally. Another spring (not shown) may also be used to bias the traveller 540 distally.

The slider 520 has at least one radially-outwardly directed external key 522 for sliding engagement with a branched keyway 542 on the inner wall of the traveller 540. The branched keyway 542 on the traveller 540 has an angled portion 544 where the external key 522 is first located prior to use of the guard 500. The angled portion 544 is for effecting axial and rotational proximal movement of the slider 520, simulating a cam-like function. This occurs when the slider 520 is pressed against the membrane of a vial to allow the needle to extend beyond the distal end 525 of the slider 520 into the vial for charging a syringe attached to the guard 500. The branched keyway 542 in the traveller 540 also has an axial portion 546. The distal end of the axial portion 546 has a recess 547. The recess 547 is for engaging the external key 522 in order to lock the slider 520 with the traveller 540 after the syringe has been charged and the guard 500 is removed from the vial. This prevents proximal movement of the slider 520 with respect to the traveller 540. The slider 520 and the traveller 540 thus function as a single unit once the external key 522 is engage in the recess 547. The recess 547 is preferably a through hole in the traveller 540.

In the same way, the traveller 540 has at least one radially-outwardly directed external key for sliding engagement with a branched keyway in the housing 560. Like the branched keyway 542 in the traveller 540, the branched keyway in the housing 560 has an angled portion for effecting axial and rotational proximal movement of the traveller 540 together with the slider 520, simulating a cam-like function. This occurs when the slider 520 is next pressed against the skin of a patient to allow the needle to extend beyond the distal end 525 of the slider 520 and penetrate the skin for injection. The branched keyway in the housing 560 also has an axial portion that has a recess 567 at its distal end for engaging the external key on the traveller 540. This locks the traveller 340 and the slider 520 with the housing 560 after injection, and prevents further proximal movement of the traveller 540 and the slider 520, so that the needle cannot be reused.

Whilst there has been described in the foregoing description exemplary embodiments of the present invention, it will be understood by those skilled in the technology concerned that many variations in details of design, construction and/or operation may be made without departing from the present invention.

1. A safety guard for a needle of a syringe, the safety guard comprising: a moveable slider for slideably encasing the needle;
a moveable traveller for sliding engagement over and with the slider and sliding engagement with and within a housing;
at least one resilient member within the housing for biasing the slider distally.

2. The safety guard of claim 1, wherein the traveller is attachable to the slider.

3. The safety guard of claim 2, wherein the traveller is attachable to the slider by radially directed projections engageable in recesses to allow distal movement and prevent proximal movement of the traveller with respect to the slider when the traveller is attached to the slider.

4. The safety guard of claim 3, wherein the projections are configured to be a snap fit in the recesses.

5. The safety guard of claim 3, wherein the projections comprise at least one cantilever snap on the traveller and the recesses comprise corresponding recesses in the slider.

6. The safety guard of claim 5, wherein the recesses in the slider comprise through holes in the slider each having a distal side angled to provide a distally sloping surface.

7. The safety guard of claim 5, wherein the recesses in the slider comprise proximal recesses and distal recesses, the proximal recesses and the distal recesses being axially spaced along the slider.

8. The safety guard of claim 7, wherein the cantilever snap on the traveller has an angled distal face such that the traveller is allowed to move distally along the slider from the proximal recesses to the distal recesses while the traveller is prevented from moving proximally when the cantilever snap is engaged with a recess.

9. The safety guard of claim 3, wherein the projections comprise at least one cantilever snap on the slider and the recesses comprise corresponding recesses in the traveller.

10. The safety guard of claim 9, wherein the recesses in the traveller comprise rectangular through holes in the traveller each having a proximal side angled to provide a proximally sloping surface.

11. The safety guard of claim 9, wherein the recesses in the traveller comprise proximal recesses and distal recesses, the proximal recesses and the distal recesses being axially spaced along the traveller.

12. The safety guard of claim 11, wherein the cantilever snap on the slider has an angled proximal face such that the slider is allowed to move proximally along the traveller from the distal recesses to the proximal recesses while the traveller is prevented from moving proximally when the cantilever snap is engaged with a recess.

13. The safety guard of claim 3, wherein the projections comprise an annular snap on the traveller and the recesses comprise annular recesses in the slider.

14. The safety guard of claim 3, wherein the projections comprise an annular snap on the slider and the recesses comprise annular recesses in the traveller.

15. The safety guard of claim 1, wherein the traveller is restrainable within the housing by further projections that engage in further recesses to allow distal movement and prevent proximal movement of the traveller with respect to the housing when the traveller is proximally restrained within the housing.

16. The safety guard of claim 15, wherein the further projections are configured to be a snap-fit in the further recesses.

17. The safety guard of claim 15, wherein the further projections comprise at least one further cantilever snap on the traveller and the further recesses comprise corresponding recesses on the housing.

18. The safety guard of claim 17, wherein the recesses in the housing comprise through holes in the housing.

19. The safety guard of claim 17, wherein the recesses in the housing are axially spaced along the housing.

20. The safety guard of claim 19, wherein the further cantilever snap on the traveller has an angled distal face such that the traveller is allowed to move distally within the housing from one recess to an adjacent distal recess in the housing while the traveller is prevented from moving proximally when the cantilever snap is engaged with a recess.

21. The safety guard of claim 15, wherein the further projections comprise an annular snap on the traveller and the further recesses comprise corresponding annular recesses in the housing.

22. The safety guard of claim 1, wherein the slider further comprises lateral protrusions for engaging proximally adjacent portions on the traveller to prevent proximal movement of the slider.

23. The safety guard as claimed in claim 15 when appended to claim 3, wherein the projections and the further projections are axially aligned.

24. The safety guard of claim 1, further comprising a cap for retaining at least a portion of the slider and at least a portion of the traveller within the housing.

25. The safety guard of claim 1, wherein the slider has at least one external key for sliding engagement with a branched keyway in the traveller.

26. The safety guard of claim 25, wherein the branched keyway in the traveller comprises an angled portion for effecting axial and rotational proximal movement of the slider, and an axial portion having a recess for retaining the external key on the slider to lock the slider with the traveller.

27. The safety guard of claim 1, wherein the traveller has at least one external key for slideable engagement with a branched keyway in the housing.

28. The safety guard of claim 27, wherein the branched keyway in the housing comprises an angled portion for effecting axial and rotational proximal movement of the traveller, and an axial portion having a recess for retaining the external key on the traveller to lock the traveller with the housing.

29. The safety guard of claim 1, further comprising a second resilient member for biasing the traveller distally.

30. The safety guard of claim 1, wherein a proximal portion of the housing is adapted for engaging the syringe.
